(19) United States
(12) Patent Application Publication
(10) Pub. No.: US 2011/0011916 A1
(43) Pub. Date: Jan. 20, 2011

(54) ANASTOMOSIS DEVICE

(75) Inventor: Jamie P. LEVINE, Scarsdale, NY (US)

Correspondence Address: NIXON PEABODY LLP - PATENT GROUP, 1100 CLINTON SQUARE, ROCHESTER, NY, 14604, US

(73) Assignee: NEW YORK UNIVERSITY, New York, NY (US)

(21) Appl. No.: 12/833,696

(22) Filed: Jul. 9, 2010

Related U.S. Application Data
(60) Provisional application No. 61/225,990, filed on Jul. 16, 2009.

(51) Int. Cl. A61B 17/115

(52) U.S. Cl. 227/179.1

(57) ABSTRACT

The present invention is directed to a circular anastomosis surgical stapler and methods of use. The circular anastomosis surgical stapler includes an operator control assembly, a tubular shaft having a distal end that is associated with a head portion, and an actuating head portion. The actuating head portion includes a detachable anvil portion with an axially extending stem portion. The anvil portion is engagable with the remainder of the head portion, which receives an array of staples. The head portion also includes an associated stapling portion with a stapling mechanism for firing an array of staples against the anvil portion. The anvil portion and the head portion matingly engage along a mating edge. The mating edge is defined by locations around the stem portion spaced from a particular position on the axis of the stem portion at axially and/or radially varying distances.

This application claims the benefit of U.S. Provisional Patent Application Ser. No. 61/225,990, filed Jul. 16, 2009, which is hereby incorporated by reference in its entirety.

### FIELD OF THE INVENTION

The present invention relates to an anastomosis device.

### BACKGROUND OF THE INVENTION

Various types of surgical fastener applying instruments are known for the application of surgical fasteners to tissue. A common type of surgical fastener is a surgical staple. Surgical stapling instruments typically have a mechanism for firing a plurality of staples from a staple holding cartridge and an anvil positioned opposite the staple cartridge against which the staples are formed. Such instruments include, for example, linear staplers, which typically apply one or more parallel rows of staples, and circular staplers, which typically apply one or more concentric/circular rows of staples. In use, the surgeon will place tissue between the staple cartridge and anvil and, by firing the instrument, cause the staples to become clenched to the tissue.

Circular staplers are known and have been successfully used in surgical procedures for many years. Commercially available instruments include: the CEEA® circular stapler, manufactured by United States Surgical Corporation, Norwalk, Conn., and the ILS®circular stapler manufactured by Ethicon, Inc., Blue Ash, Ohio. Various embodiments of circular staplers have been disclosed in U.S. Pat. No. 4,576,167 to Noiles, U.S. Pat. No. 4,603,693 to Conta et al., U.S. Pat. No. 5,005,749 to Aranyi, and U.S. Pat. No. 5,119,983 to Green et al. These instruments are typically indicated for use in gastric and esophageal surgery where tubular organs are joined to other anatomical structures. End-to-end anastomosis (“EEA”) devices are also known with a mounting means which can receive a trocar for making holes in tissue and, subsequently, an anvil shaft for the mounting of an anvil assembly for stapling purposes.

The techniques of resecting a segment of the colon or intestines and an anastomosis of the cut ends are common procedures where a segment of diseased bowel must be removed for reasons such as inflammation, oncological process, obstructions, bleeding, perforation, trauma, etc.

A conventional procedure for resection and anastomosis operates as follows. The first step is mobilization of a segment of the intestine to be resected. Mobilization is achieved by freeing the intestine from its cavity attachment and is then followed by separating its blood supply. The mobilization can be conducted laparoscopically or through opening the peritoneal cavity. Second, resection of the segment to be removed follows the mobilization procedure. Following the resection of the segment to be removed, the remaining ends of the bowel are anastomized to guarantee the continuity of the intestinal track. In an intestinal end-to-end anastomosis, a portion of the intestinal tract is removed (i.e., due to the presence of disease, such as cancer) and the remaining ends are rejoined by using a circular stapler. To join the tubular structures, one end of the intestine is secured about an anvil and the other end of the intestine is held in place adjacent a staple cartridge. Preferably, the anvil has a shaft that is removably connected to the instrument. Once the anvil shaft is secured to the instrument, the anvil is drawn into close approximation to the stapling cartridge. The instrument is then fired to cause the staples to pass through tissue of both organs and become formed against the anvil. During the firing step, a circular knife is advanced to cut tissue inside the staple line, thereby establishing a passage between the organs. After firing, the instrument is typically removed by withdrawing the anvil through the staple line, after which the surgeon will carefully inspect the surgical site to ensure a proper anastomosis (joining) has been achieved.

In a further circular stapling example, U.S. Pat. No. 5,392,979 to Green et al., discloses a stapling apparatus which permits the anvil to be attached to, or detached from, the head with comparative ease, but in which the anvil is no longer detachable from the head if the distance between the two members has reached an optimal value for anastomosis. Then, a safety mechanism is released, whereupon it becomes possible to fire the staples. An indicator is used to provide a visual signal for the surgeon to know an optimal value of the head-to-anvil distance. A similar type of suturing apparatus is described in U.S. Pat. No. 5,205,459 Brinkerhoff et al.

There are two types of existing staplers, namely, linear and circular. The presently available stapling devices generally include gastrointestinal anastomosis (“GIA”) and EEA staplers which can be used to transect tissue in linear and circular fashions, respectively, with subsequent anastomosis using staples. The linear GIA is relatively versatile. The prior art further notes that the EEA is primarily suited for lower colonic circular anastomosis after a lesion has been surgically removed (via laparotomy or laparoscopically) or during a colostomy takedown procedure. Examples of prior devices can be found in U.S. Pat. No. 4,576,167 to Noiles, U.S. Pat. No. 5,156,614 to Green et al., U.S. Pat. No. 5,170,925 to Madden et al., U.S. Pat. No. 5,172,845 to Tejeiro, U.S. Pat. No. 5,180,092 to Crainich, U.S. Pat. No. 5,188,274 to Moeinzadeh et al., U.S. Pat. No. 5,188,638 to Tzakis, U.S. Pat. No. 5,197,648 to Gringold et al., U.S. Pat. No. 5,197,649 to Bessler et al., U.S. Pat. No. 5,217,472 to Green et al., U.S. Pat. No. 5,219,111 to Bilotti et al., U.S. Pat. No. 5,220,928 to Oddsen et al., U.S. Pat. No. 5,221,036 to Takase, U.S. Pat. No. 5,242,457 to Akopov et al., U.S. Pat. No. 5,441,508 to Bessler et al., U.S. Pat. No. 6,279,809 to Nicolo, and U.S. Pat. No. 6,981,979 to Nicolo, U.S. Pat. No. 6,769,590 to Vresh et al., U.S. Pat. No. 6,945,444 to Gresham et al., and U.S. Pat. No. 7,364,060 to Milliman.

A common problem in anastomosis procedures is that such procedures are limited to straight line incisions, which heal in a constricted fashion. The resultant circular or ring-like scar may lead to narrowing of the lumen and subsequent stricture. Thus, patients are often plagued by the long-term complication of stricture around the suture or staple line of the anastomosis, e.g., anastomotic colorectal stenosis. Stricturing often leads to intestinal obstruction, perforation, and morbidity. This complication requires reoperation, stricturoplasties, and dilations. See Barba et al., “Endoscopic Dilation of Gastroesophageal Anastomosis Stricture After Gastric Bypass,” Surg. Endosc. 17:416-420 (2003).

The present invention is directed to overcoming these and other deficiencies in the art.

### SUMMARY OF THE INVENTION

One aspect of the present invention is an anastomotic surgical stapling device comprising an operator control assembly, a tubular shaft longitudinally extending from the operator control assembly to a distal end, and an actuating head portion at the distal end of the tubular shaft. The actuating head portion includes a detachable anvil portion with an axially extending stem portion and a stapling portion comprising a stapling mechanism for firing an array of staples around the stem portion and against the detachable anvil portion. The anvil portion and the stapling portion matingly engage along a mating edge defined by locations around the stem portion spaced from a particular position on the axis of the stem portion by axially and/or radially varying distances.

Another aspect of the present invention is a detachable head portion of a surgical stapling device. The detachable head portion comprising a detachable anvil portion with an axially extending stem portion and a stapling portion including a stapling mechanism for firing an array of staples around the stem portion and against the detachable anvil portion. The anvil portion and the stapling portion matingly engage along a mating edge defined by locations around the stem portion spaced from a particular position on the axis of the stem portion by axially and/or radially varying distances.

Yet another aspect of the present invention is a method of performing a surgical resectioning and restructuring for a tubular body lumen comprising the step of providing an anastomotic surgical device. This device includes an operator control assembly, a tubular shaft longitudinally extending from the operator control assembly to a distal end, and an actuating head portion at the distal end of said tubular shaft. The actuating head portion further comprises a detachable anvil portion with an axially extending stem portion and a stapling portion comprising a stapling mechanism for firing an array of staples around the stem portion and against the detachable anvil portion. The anvil portion and the stapling portion matingly engage along a mating edge defined by locations around the stem portion spaced from a particular position on the axis of the stem portion by axially and/or radially varying distances. The anastomotic surgical device is inserted through the body lumen with the longitudinal extent of the elements of the device generally parallel with the axis of the tubular body lumen. The anastomotic surgical device is actuated within the body lumen and then withdrawn.

The anastomosis surgical staplers of the present invention are similar to existing end to end anastomotic staplers, commonly called “EEA staplers,” such as those shown in U.S. Pat. No. 4,646,745 to Noiles, which is hereby incorporated by reference in its entirety. However, the head portion differs greatly from other EEA anastomosis stapling devices, because the present invention employs varying mating edges and staple lines, resulting in a w-plasty style anastomosis (i.e., an anastomosis in which the edges of a wound are trimmed in the shape of a W and closed in a zigzag fashion). In this way, narrowing of the lumen and subsequent stricture is avoided, which is a significant advantage over prior EEA devices and anastomosis methods. A typical application of the surgical stapler of the present invention is connecting together two sections of the hollow tubular body organ (e.g., two intestinal sections) by means of an array of staples, which surrounds a lumen or passageway between the interiors of the connected organ sections.

Surgically, W-plasties are commonly used for reconstructive purposes, to break up straight-line incisions and change the direction of wound contracture into multiple vectors, which can ultimately prevent stricturing. Also, w-plasty or zigzag type of incisions essentially lengthens a straight-line incision. This is tremendously important in circumferential incisions, such as what is created when an end-to-end anastomosis is performed, and in linear incisions. Both types would benefit not only from the decreased risk of long-term contracture but functionally would be improved by lengthening the incision. An example of where these zigzag type of incisions normally show significant benefit are in patients with circumferential constriction bands in the extremity. W-plasty incisions are used to acutely widen the circumferential contracture and this helps to prevent the recurrence of the contracture around the extremity. Also, this technique has been used to repair stenotic strictures related to intestinal stomas. The stricture in these circular stomas was refractory to other standard repair techniques such as dilatation. W-plasty offered an acute and long-term solution to this problem.

A potentially devastating complication of performing an intestinal anastomosis is a stricture. This complication can lead to obstruction, and perforation and depending on its location may not even be surgically repairable. The best method of dealing with these strictures is prevention. Planned placement of offset, zigzag type incisions will act to acutely expand the anastomotic opening, and chronically help to prevent circular stricturing because of the w-plasty incision placement. Traditional, circular end-to-end anastomotic devices usually leave the intraluminal diameter of the anastomosis smaller because of the need to keep the size of the anvil and coupler portions smaller than the size of the lumen being anastomed. Also, the staple portion of the anastomosis is inverted into the lumen also decreasing the functional size of the anastomosis. When combining this acute narrowing with the chronic shortening from wound contracture the intestine has a higher risk of stricturing. An acute widening of the anastomosis along with the long-term contracture prevention provided by offset incisions would be ideal for any luminal anastomosis. Beraldo et al., “Use of W-plasty in stenotic stoma: a new solution for an old problem.” Colorectal Dis. 8(8):715-6 (2006), which is hereby incorporated by reference in its entirety.

### BRIEF DESCRIPTION OF THE DRAWINGS

FIG. 1 is a partially exploded, perspective view of one embodiment of the present invention.

FIG. 2A shows an enlarged perspective view of the head portion of the instrument shown in FIG. 1. FIG. 2B is an end view of the stapling head assembly along line 2B-2B of FIG. 2A. FIG. 2C is an end view of the anvil portion along line 2C-2C of FIG. 1.

FIG. 3 is a cross sectional view of the head portion along line 3-3 of FIG. 2B.

FIG. 4 is a partially exploded, perspective view of a second embodiment of the present invention.

FIG. 5A depicts another enlarged perspective view of a second embodiment of the head portion of the instrument shown in FIG. 4. FIG. 5B is an end view of the stapling head assembly along line 5B-5B of FIG. 5A. FIG. 5C is an end view of the anvil portion along line 5C-5C of FIG. 4.

FIG. 6 is a cross sectional view of the head portion along line 6-6 of FIG. 5B.

FIGS. 7A-G are schematic views illustrating the surgical stapler of the present invention in use and the technique for removing the stapler upon completion of the anastomosis.

FIGS. 8A-D are schematic views of the initial stapler anastomosis made at the time of the cutting and stapling by the head assembly both extraluminally (FIG. 8A) and intraluminally (FIG. 8B) according to the first embodiment of the present invention in FIGS. 1-3. The resultant expanded stapler anastomosis is shown extraluminally (FIG. 8C) and intraluminally (FIG. 8D).

FIGS. 9A-B are schematic views of the initial anastomosis made at the time of stapling and cutting by the head assembly (FIG. 9A) according to a second embodiment of the present invention in FIGS. 4-6 and the resultant expanded anastomosis (FIG. 9B).

### DETAILED DESCRIPTION OF THE INVENTION

One aspect of the present invention is an anastomotic surgical stapling device comprising an operator control assembly, a tubular shaft longitudinally extending from the operator control assembly to a distal end, and an actuating head portion at the distal end of the tubular shaft. The actuating head portion includes a detachable anvil portion with an axially extending stem portion and a stapling portion comprising a stapling mechanism for firing an array of staples around the stem portion and against the detachable anvil portion. The anvil portion and the stapling portion matingly engage along a mating edge defined by locations around the stem portion spaced from a particular position on the axis of the stem portion by axially and/or radially varying distances.

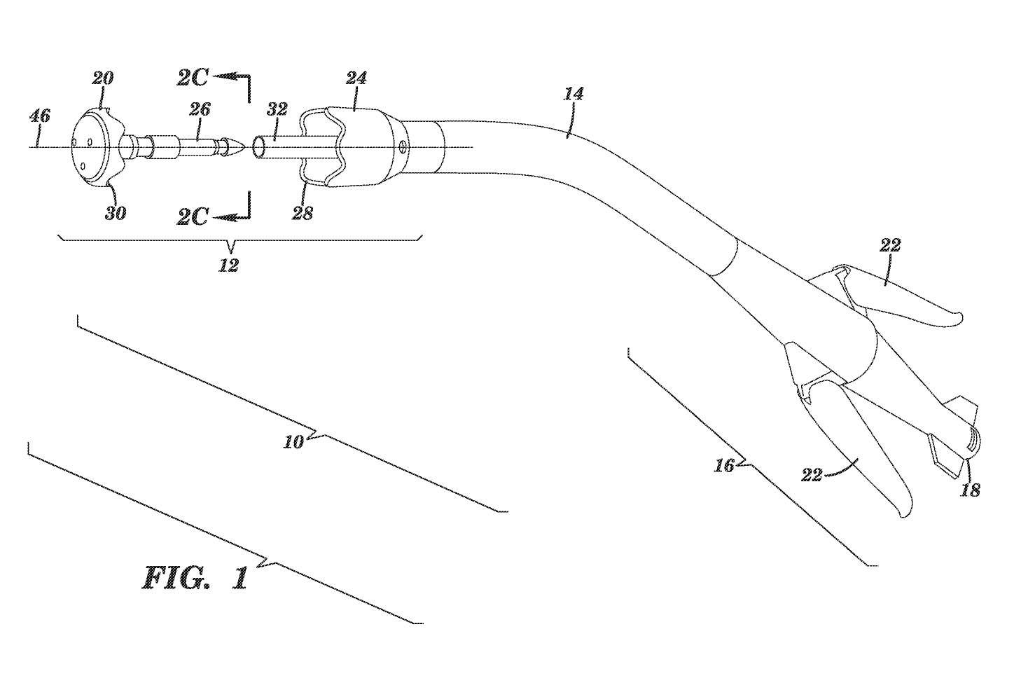

Referring now to FIG. 1, anastomosis surgical stapler 10 includes actuating head portion 12 attached to elongated tubular body 14, which may be hollow, with operator assembly 16 on the opposed end of tubular body 14 from actuating head portion 12. Elongated tubular body 14 may include a bend to better conform to the shape of the patient's bowel, other hollow viscus, or lumen.

Operator assembly 16 includes a rotary control 18 for actuating anvil 20 of stapler 10 in a conventional fashion, as will be described. Operator assembly 16 also includes trigger 22 for firing of surgical staples in a generally conventional manner, as is known in the art (e.g., U.S. Pat. No. 6,945,444 to Gresham et al. and U.S. Pat. No. 7,364,060 to Milliman, which are hereby incorporated by reference in their entirety). Operator assembly 16 may further include conventional safety lock, which acts to prevent firing of staples until anvil 20 is in the appropriate position relative to associated stapling portion 24 of actuating head portion 12. Conventional safety locks are known in the art and examples of such safety locks can be found, for instance, in U.S. Pat. No. 6,945,444 to Gresham et al. and U.S. Pat. No. 7,364,060 to Milliman, which are hereby incorporated by reference in their entirety.

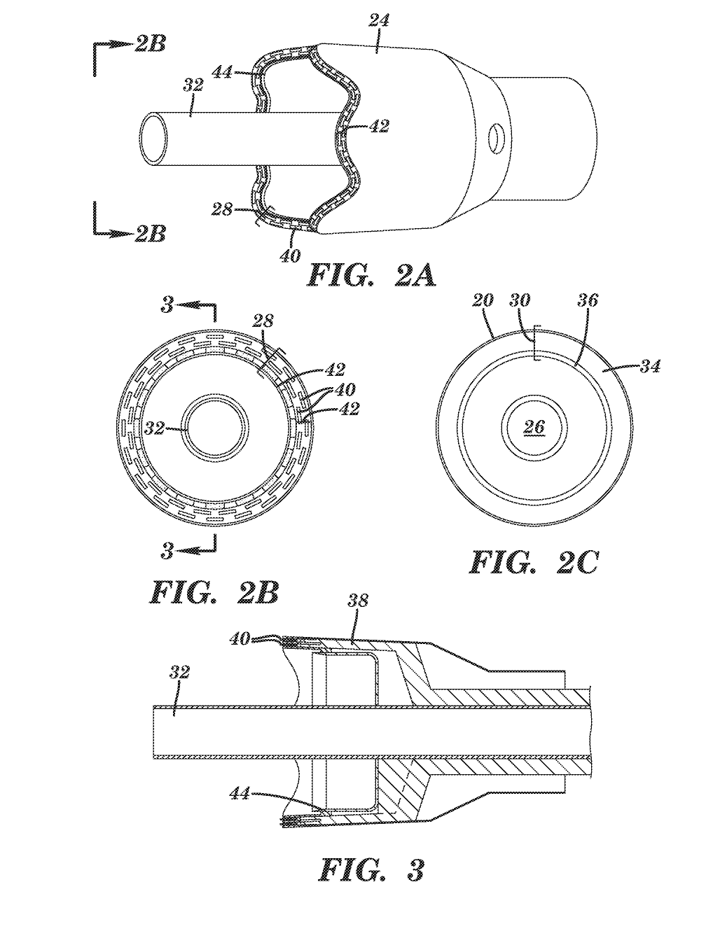

Actuating head portion 12, according to a first embodiment, is best described with reference to FIGS. 1, 2A-C, and 3. Referring to FIG. 1, actuating head portion 12 includes anvil assembly 20, mounted to the end of stem portion 26, and associated stapling portion 24. Stapling portion 24 includes stapling portion mating edge 28 and anvil portion 20 includes anvil portion mating edge 30. Stapling portion 24 also includes stem receiving device 32, which matingly engages with stem portion 26 of anvil 20 upon operation of anastomosis surgical stapler 10, as described infra. Tubular body 14 is mounted for longitudinal reciprocal motion relative to stapling portion 24 so that the spacing between opposing faces of anvil assembly 20 and stapling portion 24 may be varied. As can be seen in FIG. 2C, anvil assembly 20 includes staple anvil 34 and knife anvil or cutting ring 36.

Referring now to FIG. 3, stapling portion 24 includes staple cartridge or housing 38, which initially holds a plurality of surgical staples 40 arranged in, e.g., two closely spaced concentric annular rows 42 (See FIG. 2B). Certain embodiments may include only one concentric annular row of staples. Yet further embodiments may have more than two closely spaced concentric annular rows. The sharply pointed free ends of the legs of each staple 40 point toward staple anvil 34 of anvil assembly 20. Staple portion 24 may also include a tissue cutting knife 44, which points toward the knife anvil or cutting ring 36 of anvil assembly 20. Also included in staple portion 24 is a conventional stapling mechanism that is mounted for longitudinal motion relative to the cartridge for housing staples 38. Examples of conventional stapling mechanisms can be found, for instance, in U.S. Pat. No. 4,603,693 to Conta et al., U.S. Pat. No. 5,292,053 to Bilotti et al., U.S. Pat. No. 5,205,459 to Brinkerhoff et al., U.S. Pat. No. 6,945,444 to Gresham et al., and U.S. Pat. No. 7,364,060 to Milliman, which are hereby incorporated by reference in their entirety. The stapling mechanism is used for driving staples 40 and knife assembly 44 toward the anvil 20 upon operation of trigger 22 of operator assembly 16.

With further reference to FIGS. 2A-B and 3, the construction of the actuating head portion 12 differs greatly from other EEA anastomosis stapling devices. Unlike other circular anastomosis staplers, the anvil 20 and associated stapling portion 24 meet along varying mating edges 28 and 30 such that the anastomosis created upon operation of the device of the present invention is in a w-plasty fashion. In a first embodiment, shown generally in FIG. 1 and more specifically in FIGS. 2A-C and FIG. 3, stapling portion mating edge 28 and anvil portion mating edge 30 are axially varying such that when anvil 20 and associated stapling portion 24 mate during operation of the device, mating edges 28, 30 interdigitate parallel to longitudinal axis 46 of stem portion 26.

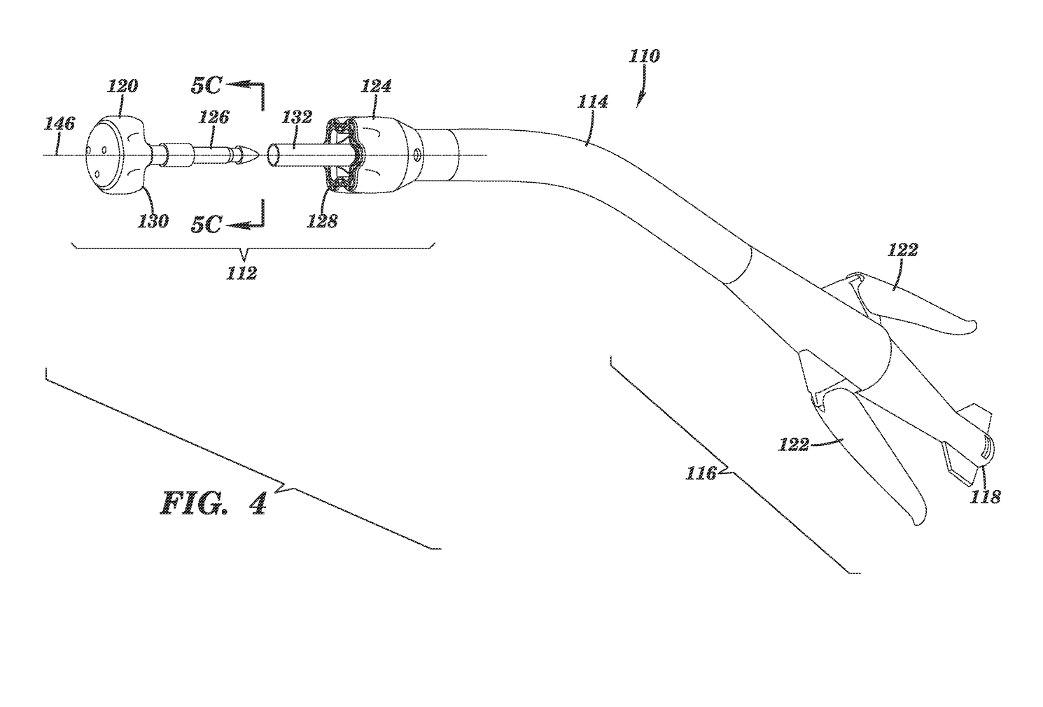

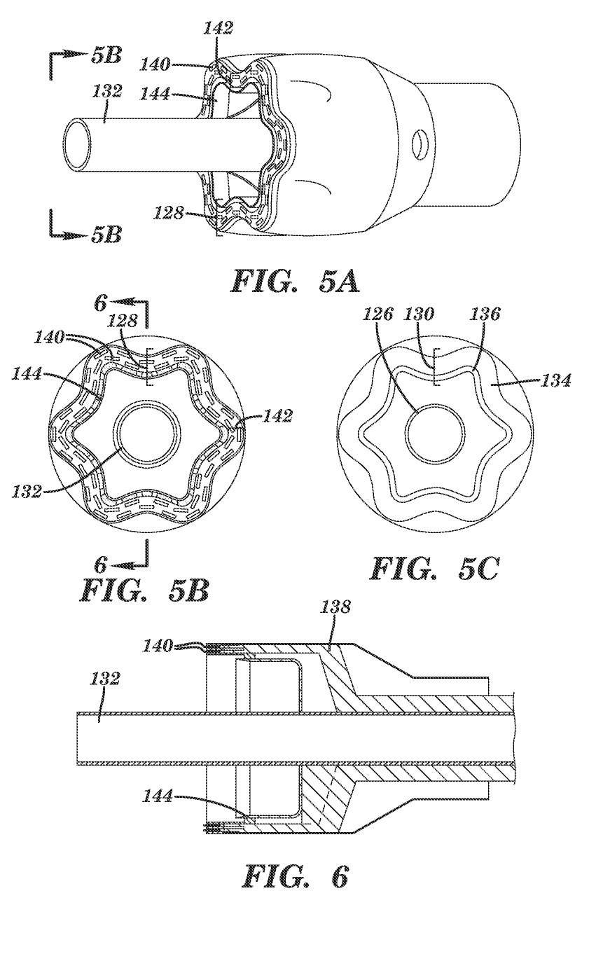

Anastomosis surgical stapler 110, according to a second embodiment of the present invention, is described with reference to FIGS. 4, 5A-B, and 6. Referring now to FIG. 4, actuating head portion 112 includes anvil assembly 120 mounted to the end of stem portion 126 and associated staple portion 124. Stapling portion 124 includes a stapling portion mating edge 128 and anvil portion 120 includes an anvil portion mating edge 130. Stapling portion 124 also includes stem receiving means 132, which matingly engages with stem portion 126 of anvil 120 upon operation of anastomosis surgical stapler 110, as described below. Tubular body 114 is mounted for longitudinal reciprocal motion relative to stapling portion 124 so that the spacing between opposing faces of anvil assembly 120 and stapling portion 124 can be varied. As can be seen in FIG. 5C, anvil assembly 120 includes staple anvil 134 and knife anvil or cutting ring 136.

Referring now to FIG. 6, stapling portion 124 includes a staple cartridge or housing 138, which initially holds a plurality of surgical staples 140 arranged in, e.g., two closely spaced concentric annular rows 142 (See FIG. 5B). Certain embodiments may include only one concentric annular row of staples. Yet further embodiments may have more than two closely spaced concentric annular rows. The sharply pointed free ends of the legs of each staple 140 point toward staple anvil 134. Staple portion 124 may also include tissue cutting knife 144, which points toward knife anvil or cutting ring 136. Also included in staple portion 124 is a stapling mechanism that is mounted for longitudinal motion relative to the cartridge for housing staples 138. The stapling mechanism is used for driving staples 140 and knife assembly 144 toward the anvil 120 upon operation of trigger 122 of operator assembly 116.

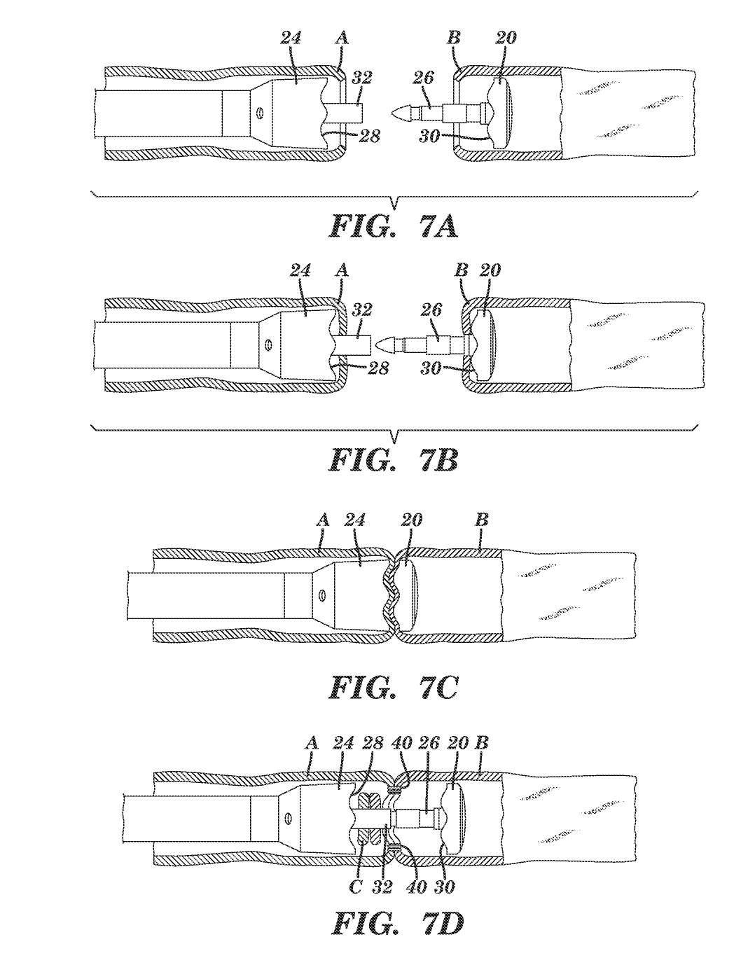

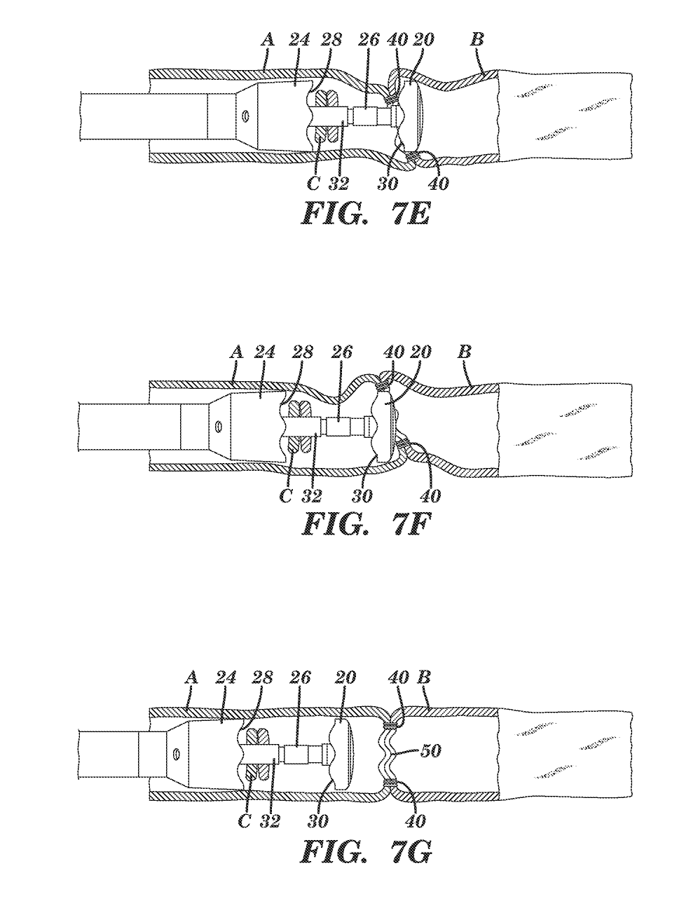

With further reference to FIGS. 5A-B and 6, the construction of actuating head portion 112 differs greatly from other EEA anastomosis stapling devices. Unlike other EEA staplers, anvil 120 and associated stapling portion 124 meet along varying mating edges 128 and 130 such that the anastomosis created upon operation of the device of the present invention is in a w-plasty fashion. In the second embodiment, shown generally in FIG. 4 and more specifically in FIGS. 5A-B and FIG. 6, stapling portion mating edge 128 and anvil portion mating edge 130 are radially varying such that when anvil 120 and associated stapling portion 124 mate during operation of the device (as illustrated in FIGS. 7A-D and described generally above), mating edges 128, 130 matingly engage along a radially varying, planar mating edge, parallel to longitudinal axis 146 of stem portion 126.

Varying mating edges according to the present invention may include any type of variation including, e.g., generally wave like, sinusoidal, sawtooth, zig-zag, or repeating square pattern. The varying edge may be symmetrical or asymmetrical.

The reference to staples and staplers in the present invention should be construed broadly to encompass the use of compression rings. Circular compression rings may also be used in anastomotic procedures such as those described in U.S. Pat. No. 6,981,979 to Nicolo et al., which is hereby incorporated by reference in its entirety. The known compression rings are two ring members that clamp tightly together with the tissue to be anastomized therebetween. The rings hold the tissue tightly together, like staples, until the tissue heals. Currently these rings are made to be eventually absorbed by the body after healing, again similar to the staples often used in anastomotic procedures. These known circular rings suffer the same disadvantages as the circular staplers discussed above.

One embodiment of the present invention provides forming the compression ring members with a wave-like shape. The wave-like shape of the compression ring members will provide the same advantages as discussed above for the staples 40 in circular staplers 10 and 110. If formed of a non-bio-absorbable material, it is apparent that any compression ring would need to either stay permanently at the anastomotic site or pass through the anastomized lumen following healing. The holding of the compression ring at the anastomotic site permanently is generally not desired and may need additional elements to integrate the ring into the healed lumen. With the current ring member configurations, the ring would be expected to naturally separate from the healed lumen. The wave-like shape of the compression ring members of the present invention (creating a w-style anastomosis and, hence, an expanded anastomosis margin) allow the ring to be more easily passed through the healed lumen. It is expected that the wave-like shaped compression ring members of the present invention will be placed into a lumen for the anastomosis. After the lumen is healed, the ring (if formed of a non-bio-absorbable material) will naturally separate from the healed lumen and simply pass through the lumen.

In an alternative embodiment of the present invention, the compression rings discussed above may be formed as frangible components that will effectively disintegrate into smaller components when the anastomosis site has healed and the ring separates from the anastomotic site to pass through the lumen as described in U.S. Pat. No. 6,981,979 to Nicolo et al., which is hereby incorporated by reference in its entirety. The object may be accomplished by forming connecting ring member segments or break lines of biodegradable material connecting adjacent individual sections of the ring member formed of non-bioabsorbable material. Alternatively, the non-absorbable ring member section may be connected to each other with a weakened portion of the ring member (e.g., a groove or slot or minimum member thickness therebetween). In this construction, the separation of the ring from the anastomotic site will result in the disintegration of the ring members into individual sections.

Circular shaped compression discs may also be used with the present invention, such as those described in U.S. Pat. No. 6,981,979 to Nicolo et al., which is hereby incorporated by reference in its entirety. These compression discs would also have a wave-like shape similar to that of the compression rings as discussed above, which would achieve a w-plasty anastomosis of the present invention.

In certain open procedures, the position of the lumen within the operating environment can make it difficult to attach stem 26, 126 of anvil 20, 120 to stapling portion 28, 128 in operations where anvil 20, 120 is attached to stapling portion 28, 128 at the anastomosis site. In order to simplify this problem, anvil 20, 120 according to the present invention may be modified with a telescoping extendable stem, which is known in the art. Such a telescoping stem is described in U.S. Pat. No. 6,279,809 to Nicolo, which is hereby incorporated by reference in its entirety.

Embodiments of the present invention may also be used with a trocar assembly, which may be used to, e.g., make holes in tissue before operation of the device with the anvil assembly. Embodiments of such a trocar assembly are shown in U.S. Pat. No. 5,119,983 to Green et al., which is hereby incorporated by reference in its entirety. Further embodiments of the present invention may incorporate an endoscopic device, such as those described in U.S. Pat. No. 5,411,508 to Bessler et al., which is hereby incorporated by reference in its entirety.

Another aspect of the present invention is a detachable head portion of a surgical stapling device. The detachable head portion comprising a detachable anvil portion with an axially extending stem portion and a stapling portion including a stapling mechanism for firing an array of staples around the stem portion and against the detachable anvil portion. The anvil portion and the stapling portion matingly engage along a mating edge defined by locations around the stem portion spaced from a particular position on the axis of the stem portion by axially and/or radially varying distances.

Yet another aspect of the present invention is a method of performing a surgical resectioning and restructuring for a tubular body lumen comprising the step of providing an anastomotic surgical device. This device includes an operator control assembly, a tubular shaft longitudinally extending from the operator control assembly to a distal end, and an actuating head portion at the distal end of said tubular shaft. The actuating head portion further comprises a detachable anvil portion with an axially extending stem portion and a stapling portion comprising a stapling mechanism for firing an array of staples around the stem portion and against the detachable anvil portion. The anvil portion and the stapling portion matingly engage along a mating edge defined by locations around the stem portion spaced from a particular position on the axis of the stem portion by axially and/or radially varying distances. The anastomotic surgical device is inserted through the body lumen with the longitudinal extent of the elements of the device generally parallel with the axis of the tubular body lumen. The anastomotic surgical device is actuated within the body lumen and then withdrawn.

Operation of the anastomosis surgical stapler 10 is best described with reference to FIGS. 7A-G. These figures illustrate: the initial placement of the anvil 20 and associated stapling portion 24 of the head 12 in two sections of body lumen A and B (e.g., intestinal sections) (FIG. 7A); the securing of the free ends of the body lumen to the stem portion 26 of the anvil 20 and the stem receiving means 32 of the stapling portion 24 of head 12, respectively (FIG. 7B); the mating of the anvil 20 and stapling portion 24 of head 12, stapling, and cutting upon firing the device (FIG. 7C); the subsequent anastomosis and excised tissue C attached to the stem portion 30 and/or stem portion receiving means 32 (FIG. 7D); and a technique for removal of the anastomosis stapling device upon completion of the stapling (FIGS. 7E-G). The removal technique could also be aided by a tilting anvil portion known to those of skill in the art (e.g., U.S. Pat. No. 6,957,758 to Aranyi and U.S. Pat. No. 7,364,060 to Milliman, which are hereby incorporated by reference in their entirety).

Briefly, anastomosis surgical stapler 10 is inserted into body lumen (e.g., intestine or bowel) sections A and B to be attached. At the anastomosis site, anvil 20 is advanced to the open position (shown in FIGS. 1 and 4). The anastomosis sites of the two (e.g., bowel sections) A and B to be attached are positioned between anvil 20 and associated stapling portion 24 and anvil 20 is tightened by operation of operator assembly 16 and trigger 22 to clamp the relevant portions of the two bowel sections A and B together. Known pull strings, and the like, may be used to assure the proper positioning of the bowel sections A and B to be attached around stem 26. Trigger 22 is actuated to fire surgical staples 40 and knife 44.

More specifically, in use in the typical application of joining two intestinal sections together, the instrument is first positioned relative to the tissue, as shown in FIG. 7A, so that stapling assembly 24 is inside the end of one intestinal section A and anvil assembly 20 is inside the end of the other intestinal section B. The severed ends of organ sections A and B are secured around stem portion 26 between assemblies 20 and 24 by means of, e.g., manually applied purse-string sutures, as shown in FIG. 7B. The instrument is then operated as described below to retract anvil stem 26 and thereby draw anvil assembly 20 toward staple holding assembly 24. When anvil stem 26 is fully retracted as shown in FIG. 7C, organ sections A and B are firmly clamped together between the opposing faces of anvil assembly 20 and stapling assembly 24.

With continuing reference to FIG. 7C, when the tissue of the organ sections A and B has been clamped together, as described above, the instrument is further operated as described below to drive the array of staples 40 from stapling assembly 24, through the clamped tissue, and against staple anvil 20. Staple anvil 34 clinches the ends of staples 40 so that organ sections A, B are securely fastened together by array(s) of staples 40. The same operation of the instrument which drives the staples also drives knife 44 to cut away the waste or excess tissue C inside array of staples 40 and thereby clear the lumen between the connected organ sections. The joining of the organ sections A, B is now complete and the instrument can be removed as shown in FIGS. 7E-G. Resulting initial anastomosis 50, is in a w-plasty fashion, as shown in FIG. 7G.

Now referring to FIGS. 7E-G, to remove the instrument from the stapled tissue, anvil assembly 20 is again separated, but not necessarily detached, from stapling assembly 24 so that the tissue is no longer clamped by the instrument. The instrument is then withdrawn by pulling it out through organ section A, as shown in FIGS. 7E-G. Excess tissue C, cut away by knife 44, remains secured to stem portion 26 and/or stem receiving means 32 by, e.g., sutures so that excess tissue C is removed with the instrument.

The above operation is similar to existing EEA staplers except that the present invention provides for operation of an EEA device with varying mating edges 28 and 30, which allows for a w-plasty stapling line and cut. Existing EEA staplers do not provide for this type of attachment between bowel or hollow body lumen sections.

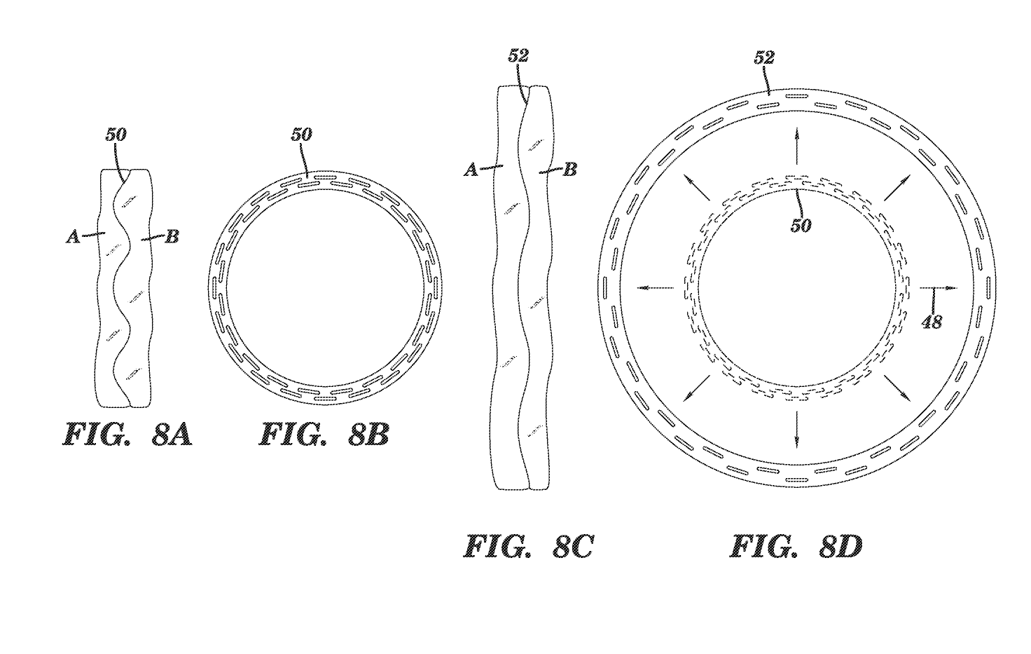

FIGS. 8A-D show the w-plasty fashion anastomosis which is achieved with circular anastomosis surgical stapler 10 of the present invention (FIGS. 1-3) after removal of the anastomosis stapler 10 as described above. FIGS. 8A and 8B are extraluminal and intraluminal illustrations of the initial anastomosis, respectively, where the initial anastomosis has an initial axially varying anastomosis margin 50. However, expansion force 48 (as a result of natural stretching, pressure exerted by body lumen contents, and removal of the circular stapling device of the present invention) drives initial anastomosis margin 50 to expanded anastomosis margin 52, relative to initial margin 50. FIGS. 8C and 8D are extraluminal and intraluminal illustrations of the expanded anastomosis, respectively.

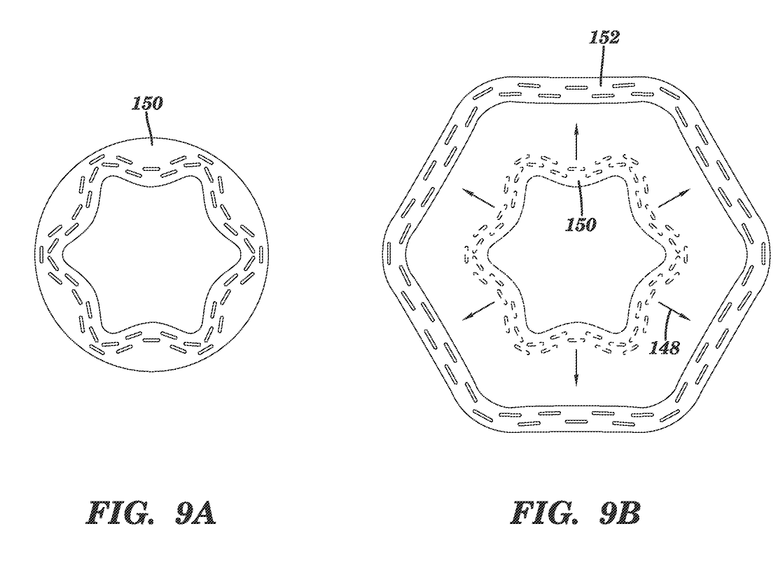

Similarly, referring now to FIGS. 9A-B, according to the second embodiment of the present invention (FIGS. 4-6), initial anastomosis 150 (as shown in intraluminal illustration, FIG. 9A) expands or stretches due to expansion force 148 to expanded, radially varying anastomosis margin 152 (as shown in intraluminal illustration, FIG. 9B).

It should be understood that embodiments of the present invention are not intended to be limited to bowel operations, but could be used for anastomosis of any hollow viscus or body lumen.

Although the present invention has been described in detail for the purpose of illustration, it is understood that such detail is for that purpose and variations can be made therein by those skilled in the art without departing from the spirit and scope of the present invention which is defined by the following claims.

What is claimed:

1. An anastomotic surgical stapling device comprising:
an operator control assembly;
a tubular shaft longitudinally extending from the operator control assembly to a distal end; and
an actuating head portion at the distal end of said tubular shaft, said actuating head portion comprising
(i) a detachable anvil portion with an axially extending stem portion and
(ii) a stapling portion comprising a stapling mechanism for firing an array of staples around said stem portion and against said detachable anvil portion, wherein said anvil portion and said stapling portion matingly engage along a mating edge defined by locations around said stem portion spaced from a particular position on the axis of said stem portion by axially and/or radially varying distances.

2. The surgical stapling device of claim 1, wherein the mating edge is in a single plane.

3. The surgical stapling device of claim 1, wherein the mating edge is in multiple planes.

4. The surgical stapling device of claim 1, wherein the mating edge is generally wave shaped.

5. The surgical stapling device of claim 4, wherein said wave shaped mating edge is in a single plane.

6. The surgical stapling device of claim 4, wherein said wave shaped mating edge is in multiple planes.

7. The surgical stapling device of claim 1, wherein said head portion further includes a knife surrounding said stem portion.

8. The surgical stapling device of claim 7, wherein said stapling portion actuates the knife when the array of staples is fired.

9. The surgical stapling device of claim 8, wherein the knife makes a radially and/or axially varying cut relative to the stem portion.

10. The surgical stapling device of claim 1, wherein the array of staples is positioned in a plane perpendicular to the longitudinal extent of said stem portion.

11. The surgical stapling device of claim 1, wherein said tubular shaft is bent along its longitudinal extent.

12. The surgical stapling device of claim 1, wherein said operator control assembly includes a mechanism for actuating said anvil portion and a trigger for firing staples in said stapling portion.

13. A detachable head portion of a surgical stapling device, said detachable head portion comprising:
(i) a detachable anvil portion with an axially extending stem portion and
(ii) a stapling portion comprising a stapling mechanism for firing an array of staples around said stem portion and against said detachable anvil portion, wherein said anvil portion and said stapling portion matingly engage along a mating edge defined by locations around said stem portion spaced from a particular position on the axis of said stem portion by axially and/or radially varying distances.

14. The detachable head portion of claim 13, wherein the mating edge is in a single plane.

15. The detachable head portion of claim 13, wherein the mating edge is in multiple planes.

16. The detachable head portion of claim 13, wherein the mating edge is generally wave shaped.

17. The detachable head portion of claim 16, wherein said wave shaped mating edge is in a single plane.

18. The detachable head portion of claim 16, wherein said wave shaped mating edge is in multiple planes.

19. The detachable head portion of claim 13, wherein said head portion further includes a knife surrounding said stem portion.

20. The detachable head portion of claim 19, wherein said stapling portion actuates the knife when the array of staples is fired.

21. The detachable head portion of claim 20, wherein the knife makes a radially and/or axially varying cut relative to the stem portion.

22. The detachable head portion of claim 13, wherein the array of staples is positioned in a plane perpendicular to the longitudinal extent of said stem portion.

23. The detachable head portion of claim 13, wherein the stem portion of said detachable anvil is received by a tube around which said stapling portion is located.

24. A method of performing a surgical resectioning and restructuring for a tubular body lumen having an axis, said method comprising the steps of:
providing an anastomotic surgical device, wherein said anastomotic surgical device comprises,
an operator control assembly;
a tubular shaft longitudinally extending distally from the operator control assembly; and
an actuating head portion at the distal end of said tubular shaft, said actuating head portion comprising:
(i) a detachable anvil portion with an axially extending stem portion and
(ii) a stapling portion comprising a stapling mechanism for firing an array of staples around said stem portion and against said detachable anvil portion, wherein said anvil portion and said stapling portion matingly engage along a mating edge defined by locations around said stem portion spaced from a particular position on the axis of said stem portion by axially and/or radially varying distances;
inserting said anastomotic surgical device through the body lumen with the longitudinal extent of the tubular shaft generally parallel with the axis of the axis of the tubular body lumen;
actuating said anastomotic surgical device within the body lumen; and
withdrawing said anastomotic surgical device from the body lumen.

25. The method of claim 24, wherein said varying mating edge is generally wave shaped.

26. The method of claim 25, wherein said wave shaped mating edge is perpendicular to the longitudinal extent of said stem portion such that said anvil portion and said stapling portion matingly engage along a planar mating edge and form a radially varying loop of staples.

27. The method of claim 25, wherein said wave shaped mating edge is parallel to the longitudinal extent of said stem portion such that said anvil portion and said stapling portion matingly engage along a non-planar mating edge and form an axially varying loop of staples.

28. The method of claim 24, wherein said head portion further includes a knife surrounding said stem portion.

29. The method of claim 28, wherein said knife is operated during said actuating such that excess tissue is excised.

30. The method of claim 28, wherein the knife makes an axially and/or radially varying cut relative to the stem portion.

31. The method of claim 28, wherein said inserting comprises:
inserting said anvil portion into a first body lumen;
inserting said stapling portion coupled to said tubular shaft and said operator control assembly in a second body lumen; and
joining said anvil portion and said stapling portion, said actuating comprising:
stapling and cutting the first and second body lumens.

32. The method according to claim 31, wherein the first and the second body lumens are from the bowel.
